(19) United States
(12) Patent Application Publication
(10) Pub. No.: US 2021/0227807 A1
(43) Pub. Date: Jul. 29, 2021

(54) METHOD FOR DETECTION AND MITIGATION OF COMPOUND PRODUCING MICROBIOTA IN AN AQUACULTURE SYSTEM

(71) Applicant: ATLANTIC SAPPHIRE IP, LLC, Miami, FL (US)

(72) Inventors: THUE HOLM, Vejle (DK); JULIAN FIORENTINO, Miami, FL (US)

(21) Appl. No.: 16/992,760

(22) Filed: Aug. 13, 2020

Related U.S. Application Data
(63) Continuation-in-part of application No. 16/991,162, filed on Aug. 12, 2020.
(60) Provisional application No. 62/964,805, filed on Jan. 23, 2020.
(60) Provisional application No. 62/985,592, filed on Mar. 5, 2020.

(51) Int. Cl. A01K 63/04; C12Q 1/689; C12N 7/00

(52) U.S. Cl. CPC A01K 63/04; C12N 7/00; C12Q 1/689

(57) ABSTRACT

A method for detecting and mitigating compound producing microbiota in an aquaculture system. The method for detecting and mitigating off-flavor producing microbiota includes, taking at least one sample of water from a portion of an aquaculture system that contains aquaculture, extracting genetic material from microbiota of the sample taken, quantifying the extracted genetic material and creating at least one bacteriophage in relation to the microbiota's genetic material, and introducing the bacteriophage(s) into the aquaculture system.

### CLAIM OF PRIORITY

This application is a Continuation-in-Part of U.S. Patent having Ser. No. 16/991,162, which was filed on Aug. 12, 2020, which claims benefit to a U.S. Provisional Patent Application having Ser. No. 62/985,592, filed on Mar. 5, 2020. Additionally, this application also claims priority to U.S. Provisional Patent Application having Ser. No. 62/964,805, filed on Jan. 23, 2020. Each of the above applications are incorporated by reference herein in their entirety.

### BACKGROUND OF THE INVENTION

### Field of the Invention

The present invention relates generally to utilizing an aquaculture system, or more specifically, a recirculating aquaculture system (RAS) for aquaculture farming and more specifically, the present invention relates to a method for detecting and mitigating microbiota that produce compounds, described as “off-flavorings” or “off-flavors,” which naturally accumulate in RAS's. Further, the present invention relates to detecting and mitigating these compound producing microbiota to ensure a desired flavor profile of aquaculture produced by a prospective RAS.

### Description of the Related Art

At the present day, a recirculating aquaculture system (RAS) is commonly used in the aquaculture farming industry. Essentially, an RAS provides an aquaculture farming production a means for a financially favorable production method, at least through limiting water consumption for use in farming. RAS's have proven to be highly effective in the art of aquaculture farming and are being adopted all over the world. Although RAS's are generally well received by the industry, when used, issues in aquaculture developing non-desired flavor profiles and RAS's experiencing unfavorable conditions are becoming more prevalent in conjunction with RAS farming. These issues are believed to be caused at least by the naturally occurring compound, Geosmin, which is an organic compound known to make water smell and taste foul to the average person. And, these issues are also somewhat believed to be caused at least by the naturally occurring compound, 2-Methylisoborneol (MIB), which is an organic chemical also known to make water smell and taste foul to the average person. It is also known that these compounds are produced by microbiota, and/or a wide range of bacteria wherein when environmental conditions are fit, the microbiota will naturally produce such compounds. Evidence for these beliefs are at least found in tasting samples of non-desirably flavored aquacultures produced in an RAS, wherein the samples of the aquacultures have been found to contain chemical concentrations of the compounds, Geosmin and 2-Methylisoborneol and in testing for specific microbiota in absence and in presence of the two compounds. Thus, it becomes apparent that these issues are becoming more prevalent in RAS's and plausibly, are known to be caused by both Geosmin and MIB. Subsequently, the two compounds have been known to be called “off-flavors” or “off-flavorings” or singularly, an “off-flavor.” In some cases, an off-flavor may also be an organic bromo-compound.

Currently, the best solution of mitigating the effects of aquaculture developing non-desired flavor profiles due to Geosmin and/or MIB, is to apply a process of depuration to the aquaculture. When aquaculture is tested and proven to have at least characteristics of “off-flavoring contamination,” depuration is carried out by purging the aquaculture in taint free water for up to 2 weeks before harvesting the aquaculture. This purging commonly requires large quantities of aquaculture to be transferred from one holding tank in an RAS to another holding tank, outside of the RAS. In some cases, purging may require holding tanks to be built once aquaculture is tested and proved to have at least characteristics of “off-flavoring contamination.” Subsequently, this process of depuration is financially taxing on aquaculture farms as the process causes the farming operation to operate outside of scheduled production via transferring and holding fish outside of their prospective RAS's and/or building costly holding tanks designed for depuration. Although this process is a partial solution, this process also creates a need to consume large quantities of clean water, which can be costly, and the aquaculture lose weight, potentially perish, or other negative side effects that would otherwise detract from the profitability of the aquaculture. Further, the process does not subsequently solve the issue of aquaculture attaining a non-desired flavor profile in farming cycles after depuration associated with the RAS, causing each following farming cycle to become contaminated and depuration still needing to be applied to the new cycles unless all contaminant are flushed from the RAS and replaced, generally by virtue of extensive deep cleaning, or replacing entire portions of the RAS, further adding to the cost. Another partial solution is deemed “Ozone Oxidation by Products,” which attempts to mitigate off-flavoring buildup/productions, but also has disparities similar to as discussed above.

As such, there is a need for a method to detect and mitigate these compounds/off-flavors before they begin affecting aquaculture in an RAS and thereby create the need for the aquaculture to go through the process of depuration and/or have the process of ozone oxidation by products applied thereto, but also a need to address a deeper root of off-flavor creation by detecting and mitigating compound producing microbiota in an RAS. Further, if the depuration process must be carried out, a need arises for a method to ensure these compounds/off-flavors will not affect aquaculture in an RAS per new farming cycles.

### SUMMARY OF THE INVENTION

In view of the foregoing disadvantages inherent in recirculating aquaculture systems now present today, the present invention provides a new method for compound detection and mitigation in an aquaculture system and a new method for detecting and mitigating compound producing microbiota in an RAS.

The general purpose of the present invention, which will be described subsequently in greater detail, is to provide a new method for compound detection and mitigation in an aquaculture system and a new method for detecting an mitigating compound producing microbiota in an RAS of which many novel features are a result, which are not anticipated, rendered obvious, suggested, or even implied by any of the prior art of aquaculture systems either in combination or singularity thereof.

Thus, the present invention is partially directed to a method to detect and/or mitigate at least one compound found in an aquaculture system, and more specifically, a recirculating aquaculture system (RAS). This method may also be known as preserving optimal flavoring of fish bred in an aquaculture system. This method may be combined with the method of detecting and mitigating compound producing microbiota in an RAS, which will be subsequently described. The method of detecting and/or mitigating at least one compound found in an aquaculture system may include, but not be limited to, taking at least one sample of water from at least one portion of the aquaculture system that contains the fish, testing the at least one sample of water to determine at least one concentration of at least one off-flavor compound therein, comparing the concentration of said off-flavor compound to an optimal concentration range, regulating the water in the aquaculture system to maintain a concentration of said off-flavor compound in the water within the optimal concentration range, and maintaining the fish within the regulated water for at least a predetermined period of time so as to reduce an amount of off-flavor that may be present in the fish.

As previously described, the aquaculture system having the method applied thereto may be a recirculating aquaculture system. The RAS may contain at least one interworking of which could be, but not be limited to, fish tanks, temperature sensors, oxygen sensors, mechanical filters, discharge pumps, backwash pumps, level sensors, reservoirs, pH sensors, pumps, lime dosing units, pumps, biological filters, trickle filters, oxygen cones, UV filters, wastewater injection systems, sludge tanks, flocculation tanks, drill strings, hatcheries, pump sumps, denitrification filters, fries and parrs, sludge concentration filters, smolt tanks, post smolt tanks, and/or on growing tanks.

As the RAS may comprise a plurality of tanks, the step of regulating the water in the aquaculture system could further comprise regulating the water within at least one tank, wherein the tank can be a tank where the fish originally resided prior to testing, or a tank alternate from where the fish originally resided prior to testing. The fish may then maintain their original location, within the now regulated tank for a period of time. In an alternative embodiment, the fish may be transferred to a regulated tank, different from where the fish originally resided and maintain their position in the regulated tank for a predetermined period of time. Further, when at least one sample of water is taken, a plurality of samples of water may also be taken from different tanks within the system and then the samples may be tested to be able to compare samples from all the tanks so as to identify variations of concentrations in the tanks. Further yet, samples of water may be taken from a plurality of different portions of the aquaculture system that contain fish at varying stages of development.

The at least one compound that the at least one sample of water may be tested for may be compounds such as, but not limited to Geosmin or 2-Methylisoborneol (MIB). These compounds may further be known as “off-flavors,” “off-flavorings,” or a single “off-flavor.” These compounds may also be known to produce non-desired flavor profiles of aquaculture, at least when the aquaculture is exposed, by means of contact, breathing, or otherwise, to the compounds, rather in singularity of just one of the compounds, or both. The concentration range that produces a non-desired flavor profile in the aquaculture may be a concentration of above 5 nanograms of Geosmin per liter of water and above 15 nanograms of MIB per liter of water when the fish are exposed to the compounds in water. A location of the RAS that contains concentrations below this range may be described as a safe limit location.

When the at least one sample of matter is tested for a concentration level (which may also be known as concentration limits) of least one compound, testing can take place via multiple different means. Testing may be carried out on a sample of matter, but not be limited to, stir bar sorptive extraction, gas chromatography-mass spectrometry, Spearman's correlation analysis of data, ANOVA analysis of data, and any combination or singularity thereof.

As water may be regulated in a tank of the RAS, and subsequently, fish maintained in the tank for a predetermined period of time of at least one week, further steps may be taken within or after the predetermined period of time. These steps may be, but not be limited to, periodically sampling a fish from within the regulated water, determining concentrations of said off-flavor compound in the sampled fish, and maintaining the fish within the regulated water until the sampled fish has concentrations of the off-flavor compounds below less than 200 nanograms of Geosmin per kilogram of fish tissue and less than 500 nanograms of MIB per kilogram of fish tissue.

Regardless of if the fish were transferred to an alternate tank, or resided in the tank they originally would have resided in prior to testing, the methodology calls to ensure the fish become located and are maintained in a safe limit location (that may contain regulated water) for at least a predetermined period of time so as to obtain and/or preserve optimal flavoring.

The maintaining of the fish in a safe limit location for at least a predetermined period of time so as to obtain and/or preserve optimal flavoring may then be followed by targeting associated compound producing sources. Targeting may be defined as identifying compound producing sources, which are generally at least one interworking of a prospective RAS. The act of targeting may also be facilitated by virtue of matching. Wherein, upon a sample being taken and subsequently tested, once the test has yielded concentration limit results, the sample and the results can then be matched back to the location it was taken at, and a user may then gain more insight as to what may be producing, if any, off-flavors. Upon this targeting and subsequently matching process, a remedying of the targeted compound producing sources from producing at least one compound may be completed. Such a targeted source(s) may be any interworking of the RAS, or, more specifically, an interworking of the RAS that has come in contact with a byproduct of the RAS, such as, but not limited to, fish waste, fish feed or sludge. This remedying may comprise avoiding or limiting clearing/cleaning of an interworking of the RAS, improving rates at which resources get added to the RAS, such as a nitrification rate, and/or adjusting the frequency of cleaning and/or backwashing an interworking of the RAS and more, again, as will be subsequently described.

The method may also include, but not be limited to, taking at least one sample of matter from at least a portion of an RAS, then testing the at least one sample of matter to determine a concentration of at least one compound in such an at least one sample of matter, and then, regulating the RAS accordingly upon determining a concentration of the at least one compound in the at least one sample of matter. In another embodiment, this may be described as, taking at least one sample of water from at least one portion of the aquaculture system that contains fish, testing the at least one sample of water to determine at least one concentration of at least one off-flavor compound therein, and regulating at least one content of the aquaculture system accordingly upon determining a concentration of the at least one sample of water. The at least one sample of matter may be, but not be limited to, matter that maintains an unfixed volume and shape, such as, but not limited to, water, aquaculture tissue, sludge, or combinations thereof.

The at least one portion of the RAS that has at least one sample of matter taken from it, may be an inlet into the RAS, an interworking of the RAS, an outlet of the RAS, or any combination or singularity of these elements. An inlet of an RAS may be defined as, any area in which the RAS receives a resource that the system will use in order to operate such as, water or aquaculture feed. An outlet may be defined as, any area in which the RAS discharges or otherwise gets rid of an unused or used resource such as, fish waste, sludge, or water. An interworking of the RAS could be any singular or combination of the elements as previously described.

As there are many possible locations for a sample of matter to be taken from a prospective RAS, at least one sample is taken. In some embodiments of the present invention, multiple samples can be taken. Multiple samples can be taken from one portion of the RAS and/or multiple samples can be taken across multiple portions of the RAS. Multiple samples may also be composed of multiple types of matter wherein the matter maintains an unfixed volume and shape. By way of non-limiting example, if two samples were taken, one sample could be a sample of matter wherein the matter is aquaculture tissue and the other sample could be water. There also may be an unlimited timeline in which samples take place. By way of non-limited example, again, if two samples were taken, one sample could be on a “day 1,” and another could be on a “day 5.”

As should be inherent, the RAS may be structured to contain, grow, and/or otherwise farm aquaculture. The aquaculture may be, but not be limited to, shrimp, lobster, scallops, salmon, tuna, sea bass, halibut, cod, jack, octopus, anchovies, crab, marlin, swordfish, mahi mahi, porgy, snapper, hog fish, ballyhoo, catfish, trout, eel, flounder, herring, tilapia, sturgeon, pikeperch, whitefish, carp, haddock, mullet, and/or mackerel.

As previously stated, the at least one compound that the at least one sample of matter may be tested for may be compounds such as, but not limited to Geosmin or 2-Methylisoborneol (MIB).

Also as previously described, when the at least one sample of matter is tested for a concentration level (which may also be known as concentration limits) of least one compound, testing can take place via multiple different means. Testing may be carried out on a sample of matter, but not be limited to, stir bar sorptive extraction, gas chromatography-mass spectrometry, Spearman's correlation analysis of data, ANOVA analysis of data, and any combination or singularity thereof.

Upon determining a concentration of at least one compound, a prospective RAS, and subsequently at least one of its contents (which may be, but not be limited to aquaculture, water, sludge or any byproduct of the aquaculture system or resource that enters the aquaculture system) may then be regulated accordingly in relation to the concentration and/or concentration level/limit the testing has yielded. Regulating an RAS accordingly may be carried out by virtue of designating aquaculture within the RAS to an appropriate area. An appropriate area may be, a portion of the RAS the aquaculture would be placed in as if the system were to continue functioning as normal, a location within the RAS but alternate to where the aquaculture was previously located within the RAS, or an isolated depuration tank. An appropriate area may also be defined as a safe limit location, or a non-safe limit location, which may be described as, but not be limited to, a warning limit or a critical limit.

The designation of aquaculture may be dependent on the concentration determined by testing. A concentration may be designated between concentration levels or limits, the first level may be thought of as a safe limit, the next level may be thought of as a warning limit, and the third level may be thought of as a critical level. A safe limit may be defined as a concentration of at least one off-flavor that will not noticeably affect the flavor profile of the aquaculture. A warning limit may also be defined as a concentration of at least one off-flavor that will not noticeably affect the flavor profile of the aquaculture, but may be nearing a quantifiable concentration value of an off-flavor that may noticeably affect the flavor profile of the aquaculture. Lastly, a critical level may be defined as a concentration of at least one off-flavor that will noticeably affect the flavor profile of the aquaculture.

In an embodiment wherein aquaculture is designated to a portion of the RAS the aquaculture would normally be placed without intervention, the RAS may then be subjected to continue functioning as normal without interruption, as will be subsequently described. This embodiment is generally followed after the testing yields results that defines aquaculture to retain their prospective desired flavor profiles, generally by virtue of the concentration testing has yielded to be designated as a safe limit. After a period of time, the entire method could then be designated as complete or be restarted.

In another embodiment, the aquaculture may be taken from a first location and placed in an alternate portion of a prospective RAS. This alternate portion of the RAS may be deemed to be a safe limit location. This safe limit location may be defined as, a portion of the prospective RAS that has at least one safe limit of a concentration of at least one off-flavor. This placement may occur following when a concentration of at least one of the off-flavors has been tested at a first location a-of the RAS and has been determined to be at a warning limit or critical limit. Following this testing and determination, is when aquaculture may be taken and placed to an alternate, safe limit location portion of the RAS.

In yet another embodiment, the aquaculture may remain in a first location following testing, even in the event that testing yielded a critical limit location. In such an embodiment, the water at the critical limit location may be regulated so as to bring in non-off-flavor contaminated water into the location and dilute the critical limit location. In this embodiment, the location may then be subsequently become a safe limit location.

This placement of aquaculture in an alternate portion of a prospective RAS or keeping of aquaculture in a first location may then be followed by targeting associated compound producing sources. Targeting may be defined as identifying compound producing sources, which are generally an interworking(s) of a prospective RAS. The act of targeting may also be facilitated by virtue of matching. Wherein, upon a sample being taken and subsequently tested, once the test has yielded at least one concentration limit, the sample and the results can then be matched back to the location it was taken at, and a user may then gain more insight as to what may be producing, if any, off-flavors. Upon this targeting and subsequently matching process, a remedying of the targeted compound producing sources from producing at least one compound may be completed. Such a targeted source(s) may be any interworking of the RAS, or, more specifically, an interworking of the RAS that has come in contact with a byproduct of the RAS, such as, but not limited to, aquaculture waste, aquaculture feed or sludge. This remedying may comprise avoiding or limiting clearing/cleaning of an interworking of the RAS, improving rates at which resources get added to the RAS, such as a nitrification rate, and/or adjusting the frequency of cleaning and/or backwashing an interworking of the RAS and more, as will be subsequently described.

In yet another embodiment, the aquaculture may be isolated via placement of the aquaculture in a depuration tank and/or putting the aquaculture through the process of depuration. Such a placement may be elicited by aquaculture existing in a first location, then that first location being sampled, tested and determined to have a critical level of a concentration of at least one off-flavor. Upon placement of the aquaculture in a depuration tank, the aquaculture is designated to remain there for a pre-determined period of time. The depuration tank may contain taint free water. Further, as the aquaculture may be designated to remain in the depuration tank for a pre-determined period of time, the depuration tank may be subject to pre-determined refreshing and/or cycling of taint free water throughout the course of aquaculture being designated to remain in the depuration tank for a pre-determined period of time. The aquaculture, after remaining in the tank for the predetermined period of time may then be sampled, by means of tasting, or by testing the chemical residue associated with the aquaculture's prospective tissue. The flavor profile may then be ensured.

This placement of aquaculture in a depuration tank and/or putting the aquaculture through the process of depuration may then be followed by targeting associated compound producing sources.

Targeting may be defined as identifying compound producing sources, which are generally at least one interworking of a prospective RAS. The act of targeting may also be facilitated by virtue of matching. Wherein, upon a sample being taken and subsequently tested, once the test has yielded concentration limit results, the sample and the results can then be matched back to the location it was taken at, and a user may then gain more insight as to what may be producing, if any, off-flavors. Upon this targeting and subsequently matching process, a remedying of the targeted compound producing sources from producing at least one compound may be completed. Such a targeted source(s) may be any interworking of the RAS, or, more specifically, an interworking of the RAS that has come in contact with a byproduct of the RAS, such as, but not limited to, aquaculture waste, aquaculture feed or sludge. This remedying may comprise avoiding or limiting clearing/cleaning of an interworking of the RAS, improving rates at which resources get added to the RAS, such as a nitrification rate, and/or adjusting the frequency of cleaning and/or backwashing an interworking of the RAS and more, again, as will be subsequently described.

As previously mentioned, the above method may be combined with the method of detecting and mitigating compound producing microbiota in an RAS. Thus, the present invention is primarily directed to a method to detect and/or mitigate at least one compound producing microbiota found in an aquaculture system, and more specifically, a recirculating aquaculture system (RAS). The method may also include, but not be limited to, taking at least one sample of matter from at least one portion of the aquaculture system, extracting genetic material from microbiota of the at least one sample, quantifying the microbiota's extracted genetic material, creating at least one bacteriophage in relation to the quantified microbiota's genetic material and introducing the at least one bacteriophage into the aquaculture system.

Again, at least one portion of the RAS that has at least one sample of matter taken from it, may be an inlet into the RAS, an interworking of the RAS, an outlet of the RAS, or any combination or singularity of these elements. An inlet of an RAS may be defined as, any area in which the RAS receives a resource that the system will use in order to operate such as, water or aquaculture feed. An outlet may be defined as, any area in which the RAS discharges or otherwise gets rid of an unused or used resource such as, fish waste, sludge, or water. An interworking of the RAS could be, but is not limited to, fish tanks, temperature sensors, oxygen sensors, mechanical filters, discharge pumps, backwash pumps, level sensors, reservoirs, pH sensors, pumps, lime dosing units, pumps, biological filters, trickle filters, oxygen cones, UV filters, wastewater injection systems, sludge tanks, flocculation tanks, drill strings, hatcheries, pump sumps, denitrification filters, fries and pans, sludge concentration filters, smolt tanks, post smolt tanks, and/or on growing tanks.

As there are many possible locations for a sample of matter to be taken from a prospective RAS, at least one sample is taken. In some embodiments of the present invention, multiple samples can be taken. Multiple samples can be taken from one portion of the RAS and/or multiple samples can be taken across multiple portions of the RAS. Multiple samples may also be composed of multiple types of matter wherein the matter maintains an unfixed volume and shape. By way of non-limiting example, if two samples were taken, one sample could be a sample of matter wherein the matter is aquaculture tissue and the other sample could be water. There also may be an unlimited timeline in which samples take place. By way of non-limited example, again, if two samples were taken, one sample could be on a “day 1,” and another could be on a “day 5.”

Again, the RAS may be structured to contain, grow and/or otherwise farm aquaculture, as described above.

A sampling of matter may take place by a multitude of different means, such as, but not limited to, an olfactory sample, observational sample, and/or a physical sample of a substance. Within an RAS, a user (as defined above) may take multiple samples of matter, across multiple locations of an RAS. By way of non-limiting example, a user may take multiple olfactory and observational samples across multiple interworkings of an RAS, then decide to take a physical sample of water from one interworking of an RAS.

As should be inherent, in order to complete the inventive methodology, at least one of the samples of matter taken must at least comprise one living organism. As such, one living organism, and/or multiple organisms bearing at least one cell will be defined as microbiota. Thus, upon taking at least one sample comprised of at least microbiota, the inventive methodology calls for extraction of genetic material from the prospective microbiota.

Extraction of genetic material from the prospective microbiota may be completed along a series of procedures across multiple means as will be subsequently described.

Upon extraction of genetic material, the extracted genetic material must be quantified. Extracted genetic material may be, but not be limited to, deoxyribonucleic acid (DNA) and/or ribonucleic acid (RNA). Quantification of such material may also be completed via a multitude of different means, which may include a series of procedures in order to complete quantification. The use of biological libraries, databases, or otherwise biological information that has/is stored and/or available may be utilized in order to supplement quantification of such material. DNA and/or RNA may first be sequenced, then annotated. The sequencing of DNA and/or RNA may take place via computation means, including utilizing software designated for sequencing of DNA/RNA. Further, the results of DNA and/or RNA annotation may have statistical analysis performed thereon to ensure correct results of annotation. Statistical analysis may be performed via computational means, including utilizing software designated for statistical analysis of DNA and/or RNA annotation.

Annotation and/or statistical analysis performed thereon may be then utilized to construct at least one genome alignment. Such a construction of at least one genome alignment may be performed utilizing computational means, and specifically, software designated to perform a genome alignment from annotated, sequenced DNA and/or RNA. A genome alignment may result from the microbiota in which DNA and/or RNA which was sampled. The at least one genome may further be utilized for creation of at least one bacteriophage, bacteriophage cocktail and/or bacteriophage therapy. Creation of at least one bacteriophage, bacteriophage cocktail and/or bacteriophage therapy may be selected from a phage genome sequence, which may either be devised by a user, and/or selected from a biological database/library. In any event, such a phage genome sequence may be identified via the ability of the phage genomes sequence direct equivalent, or near similarity to a phage genome sequence commonly known to infect the quantified microbiota's genetic material. Such a phage genome may comprise at least one quantified nucleotide sequence, which may aid in identification of genome sequence abilities. Further, in order to create at least one bacteriophage, bacteriophage cocktail and/or bacteriophage therapy, a biofoundry methodology may be applied within the inventive methodology in order to create and/or devise at least one physical bacteriophage. As such, once at least one physical bacteriophage has been created and/or devised, the at least one physical bacteriophage may then be placed in a bacteriophage cocktail and/or have a bacteriophage therapy designed and/or implemented thereon.

Upon creation and/or division of at least one physical bacteriophage, which may be placed in a bacteriophage cocktail and/or have a bacteriophage therapy designed and/or implemented thereon, in which a bacteriophage therapy may utilize at least one bacteriophage cocktail, a bacteriophage therapy may be introduced into the RAS. The inventive methodology may then be complete, or restarted.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a flowchart illustrating the overall process of the present invention of a method for compound detection and mitigation in a recirculating aquaculture system.

FIG. 2 is a flowchart illustrating a more detailed process of the present invention as show in FIG. 1.

FIG. 3 is yet another flowchart illustrating a more detailed process of the present invention as shown in FIG. 1.

FIG. 4 is yet another flowchart illustrating a more detailed process of the present invention as show in FIG. 1.

FIG. 5 is yet another flowchart illustrating an alternative embodiment of the present invention of a method of detection and mitigation of compound producing microbiota in an aquaculture system.

FIG. 6 is yet another flowchart illustrating a more detailed process and alternate embodiment of the present invention as shown in FIG. 5.

FIG. 7 is yet another flowchart illustrating another overall process of the present invention of a method for detection and mitigation of compound producing microbiota in an aquaculture system.

Like reference numerals refer to like processes throughout the several views of the drawings.

### DETAILED DESCRIPTION OF THE PREFERRED EMBODIMENT

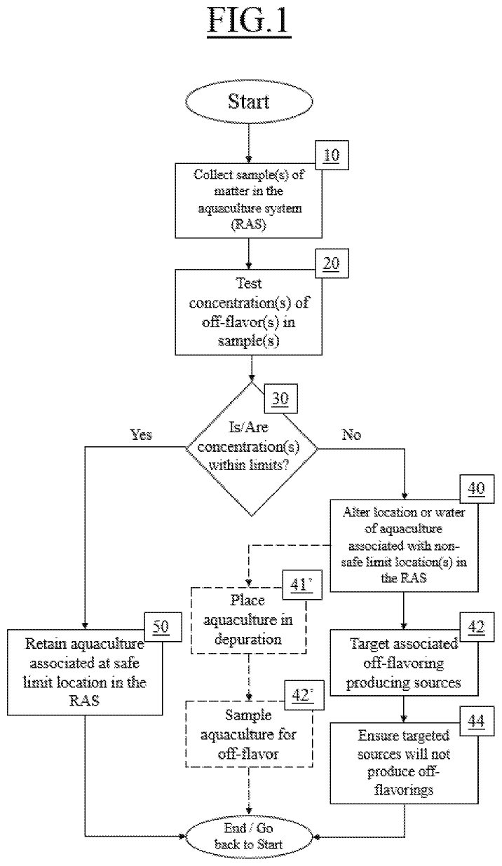

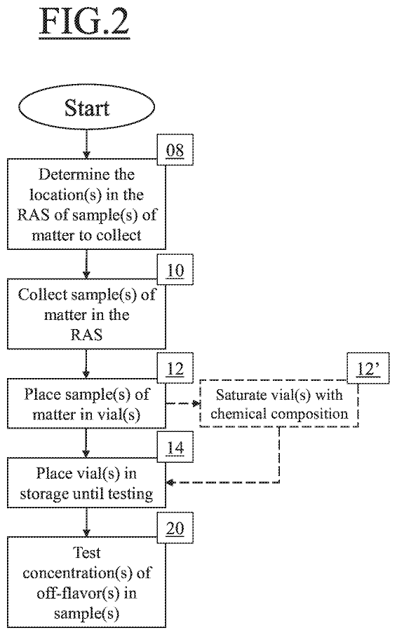

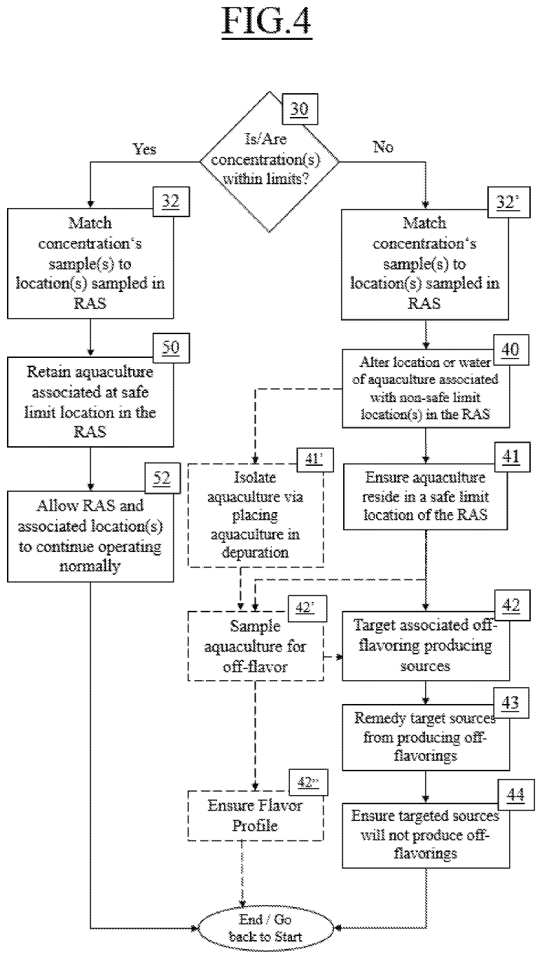

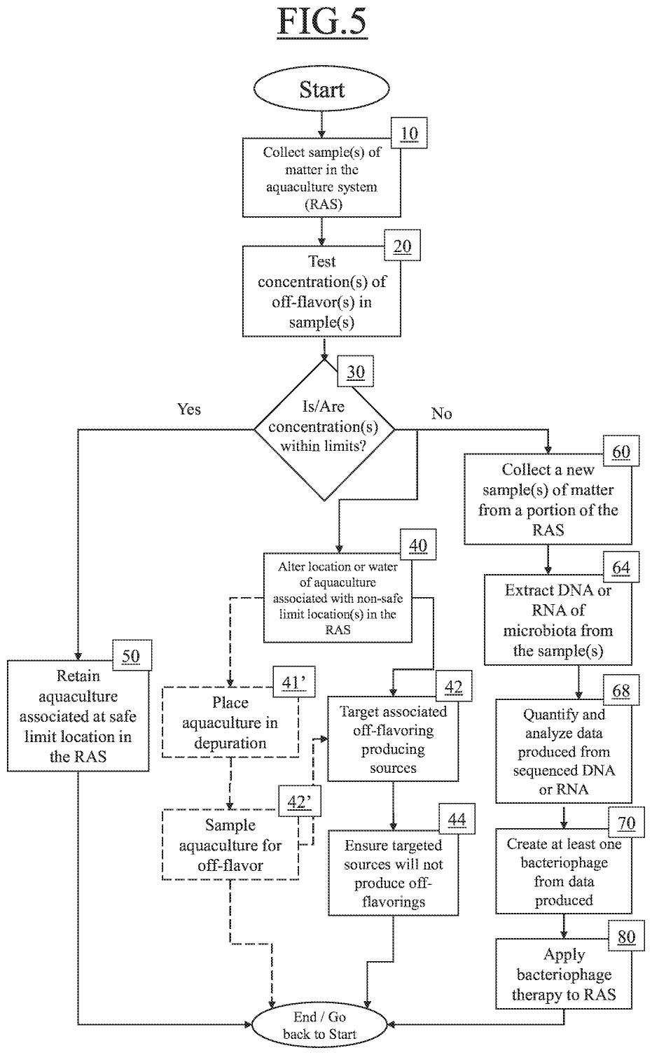

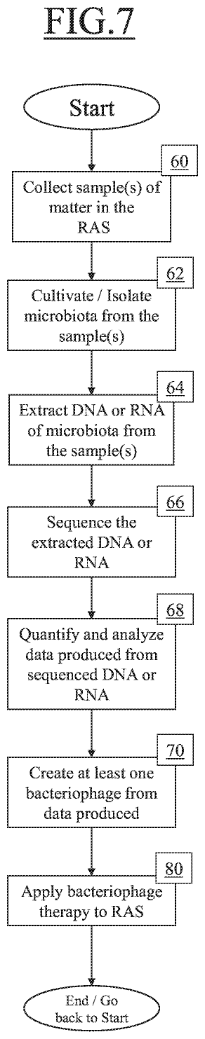

Turning now descriptively to the figures, FIG. 1 illustrates an inventive method for compound detection and mitigation in an aquaculture system from the standpoint of a general overview. The aquaculture system can be, and will be referred to as a recirculating aquaculture system. FIGS. 2, 3, and 6 will be subsequently described, and rely on FIG. 1, FIG. 4, FIG. 5 and/or FIG. 7 for context as they may be more detailed and magnified processes of the present invention described in FIG. 1, FIG. 4, FIG. 5, and/or FIG. 7.

FIG. 4 and FIG. 5 will be subsequently described, and will rely on FIG. 1 for context, as it is a more detailed and magnified process of the present invention described in FIG. 1.

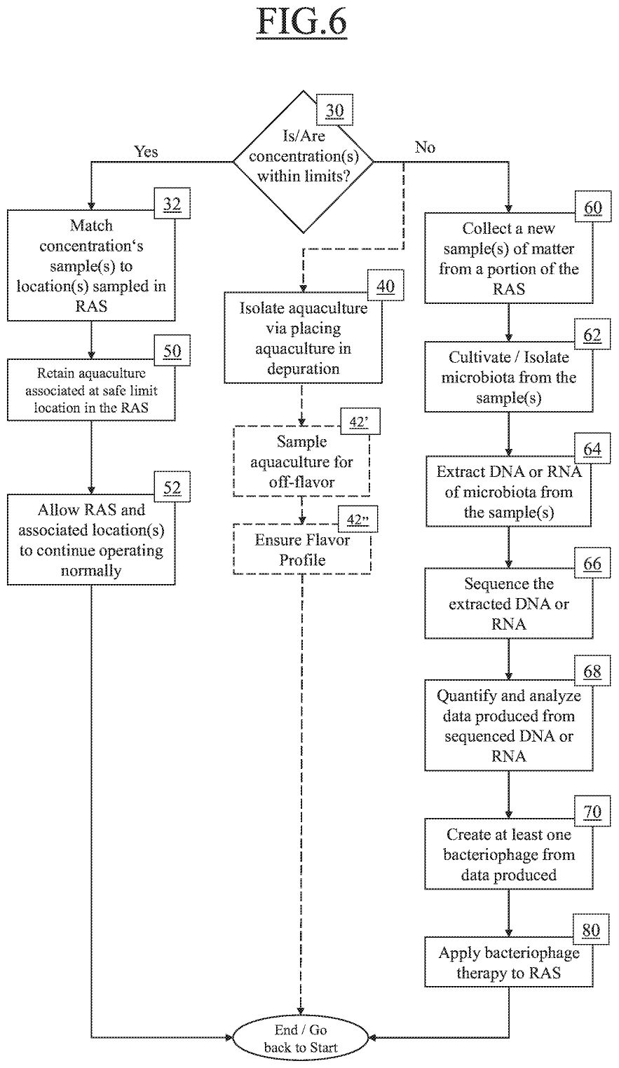

FIG. 7 illustrates an inventive methodology for detection and mitigation of compound producing microbiota in an aquaculture system and may be referred to as a general overview for the embodiments of FIG. 5 and FIG. 6.

FIGS. 1 and 2 or FIGS. 5 and 2 may be described as a starting point for the present inventive method and includes the location or locations within a recirculating aquaculture system (RAS) of a sample or samples of matter to collect 08. It may be important to note, the ambient air around an RAS or around a facility in which an RAS may be defined as a location or multiple locations as identified in procedure 08. A user, group, team, automated computer system, robot, or otherwise will complete this procedure, and subsequently most others, and will be referred to as “a user” or “the user” or “user.” A user may choose a single location or multiple locations of a recirculating aquaculture system to carry out procedure 08. Once the procedure of determining the location or locations within an RAS of a sample or samples of matter to collect 08 has been completed, the user may choose to indicate and/or record which location or locations of the respective RAS has been selected. Indication and/or recording of which location or locations of the RAS has been selected may be recorded by pen and paper, computer table entry, and/or by automated means.

Following completion of procedure 08, the user may then collect a sample or samples of matter held within the RAS 10. 10's collection of a sample or samples may be made in regards to the location or locations determined in 08. Procedure 10 may be carried out by collecting one sample of matter or multiple, wherein the user may then indicate properties of the sample or samples of matter, such as, but not be limited to, the time and date in which the sample or samples was/were taken, what the contents of the sample or samples is/are, mass or masses of the sample or samples, and/or other basic quantifiable properties of the sample or samples. The sample or samples may also be taken at a location or locations designated to different stages of aquaculture development, which, by non-limiting example, may have a sample taken at a portion of the aquaculture system designated to hold aquaculture at 1 week since birth, and another sample is taken at a portion of the aquaculture system designated to hold aquaculture at 3 weeks since birth. By way of further example, a sample or samples of ambient air around the RAS and/or around the facilities in which the RAS is housed may be taken by means of collecting air through a gas washing flask(s) using a vacuum pump and/or exposing absorbent tubes and/or traps, such as Tenax traps to such ambient air. By way of another non-limiting example, if multiple samples are taken, a user may choose to take an individual sample, or multiple samples at one point in time, and then may choose to take an individual sample, or multiple samples at another point in time.

Upon completion of procedure 10, the user may then place the sample or samples of matter in a vial or vials 12. This placing of the sample or samples of matter in a vial or vials may also be accompanied with the procedure of saturating the vial or vials with a chemical composition 12′. The vial or vials as described in procedure 12 may be composed of glass, aluminum, plastic, or any other material generally comprising vials, or any combination or singularity thereof. In one embodiment, the vial or vials may be known as a flask or flasks. In such an embodiment wherein the vial or vials are known as a flask or flasks, the flask or flasks may contain samples of a specified volume of ambient air. In an embodiment wherein the vial or vials as described in procedure 12 is/are composed of plastic, such plastics should be free from any flavor compounds and certified for collecting water samples for chemical analysis, such as, but not limited to Nalgene Bottles, fluorinated polypropylene bottles, and/or Teflon bottles. The vial or vials may also be open topped, wherein the contents of the vial are at least partially exposed to the prospective environment, or the vial or vials may be closed top, wherein the contents of the vial are sealed and unexposed to the prospective environment. The chemical composition, as described in procedure 12′ may be that of sodium chloride or any other chemical composition known for saturation at any given concentration level, from 0-99.99 percent.

After a user completes procedure 12, and possibly 12′, the user may then place the vial or vials in storage until testing 14. The procedure 14, may allow the vial or vials to be placed in a temperature controlled storage area, or a lab or environment associated with the user's testing area. If the vial or vials are to be placed in a temperature controlled storage area, the temperature may be set at a level so as to not alter the material properties of the sample or samples in the vial or vials. In an embodiment wherein the vial or vials as described in procedure 12 is/are composed of glass, a user may then place the vial or vials in storage in a temperature controlled storage area for a predetermined period of time at a temperature such as 41 degrees Fahrenheit. In an embodiment wherein the vial or vials as described in procedure 12 and above is/are composed of plastic, a user may then place the vial or vials in a storage temperature controlled storage are for a predetermined period of time at a temperature such as negative 4 degrees Fahrenheit.

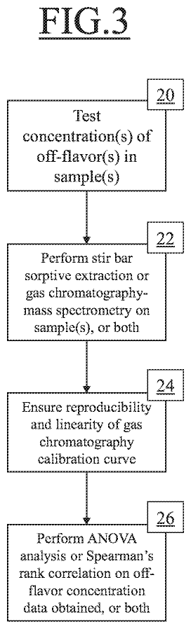

Referring now to FIGS. 1 and 3 or FIGS. 5 and 3, the procedures described within FIG. 3 will be completed following the procedures as shown and described in FIG. 2. and parts of FIG. 1 or parts of FIG. 5. A user may take the vial or vials that were placed in storage until testing, as described in procedure 14, and prepare them for testing. Preparing the vial or vials for testing may include moving the vials from storage to a prospective testing area. A user may then test for a concentration or concentrations of a compound or compounds within the sample or samples 20. In procedure 20, one example of the compounds that may be tested for may better be known as Geosmin or 2-Methlyborneol (MIB). These two naturally occurring compounds may also be referred to as “off-flavorings,” “off-flavors,” or in singularity as an “off-flavor.” Testing may also be performed so as to identify organic bromo-compounds.

The testing, as described in procedure 20 may be carried out via the methods of stir bar sorptive extraction and/or gas chromatography-mass spectrometry on the sample, or samples 22. By way of non-limiting example, the method of stir bar sorptive extraction in procedure 22 may be carried out by utilizing at least one commercial stir bar, which may be coated with polydimethylsiloxane. The stir bar or stir bars may then be placed in the vial or vials which result from procedure 12 and/or procedure 12′ wherein the vial or vials contain at least one sample. The stir bar or stir bars may then be stirred at a set revolution per minute for a specified period of time with the vial or vials. The stir bar or stir bars may then be removed via utilization of forceps, rinsed in diluted water, and dried with lint-free tissue. After drying, the stir bar or stir bars may then be transferred to thermal desorption tubes for further analysis.

By way of non-limiting example, the method of gas chromatography-mass spectrometry may then be carried out on the sample or samples following the example of stir bar sorptive extraction as described previously. This example, may take the stir bar or stir bars that had been transferred to thermal desorption tubes and a user may prepare a calibration curve or multiple calibration curves about a stir bar or stir bars from a gas chromatography mixture of Geosmin and MIB pure compounds at a multitude of dilution series, in ng/L in water. An automated thermal desorption unit may then be used to analyze the thermal desorption tubes in relation to the prepared calibration curve or calibration curves. Throughout the process of gas chromatography-mass spectrometry, a dilution or dilutions for a standard curve or curves may be analyzed in singular, duplicate, or triplicates. The standard curve or curves may also be prepared separately for each sample and/or sample type if in the process there are multiple samples and/or sample types. In the embodiment as previously described wherein ambient air was sampled, such a sample or samples may be subject also to stir bar sorptive extraction, automated thermal desorption, gas chronometry mass spectrometry, and/or a combination thereof.

Upon completion of procedure 22, a user may ensure reproducibility and linearity of the gas chromatography calibration curve or curves 24. This may be completed, by way of non-limiting example via continuing the non-limiting example as described above, of gas chromatography-mass spectrometry. A user, may analyze the calibration curve or curves that resulted from gas chromatography-mass spectrometry from a statistical standpoint. A user may quantify the data from such a calibration curve or curves and place the data in a statistical software, such as JMP. A user may also quantify alternative data that resulted from previous procedures in the method at the user's discretion to be used in conjunction with data accumulated from gas chromatography-mass spectrometry and place the data in a statistical software, such as JMP. The data may then be analyzed via assistance of statistical software such as JMP, to obtain values such as, but not be limited to, relative standard deviations or R-squared values. Upon analyzation, a user should ensure reproducibility and linearity of data to be satisfactory via obtaining relative standard deviation values of less than 3 percent and R-squared values equal to or greater than 0.98.

Following procedure 24, a user may then perform ANOVA analysis and/or a Spearman's rank correlation on the off-flavor or off-flavors concentration data obtained 26. By way of non-limiting examples via continuing the non-limiting example as described above, that of procedure 24. Following ensuring reproducibility and linearity, a user may wish to then test the data obtained from gas chromatography-mass spectrometry or data the user quantified to be used in conjunction with gas chromatograph-mass spectrometry to obtain further quantifiable data. Further quantifiable data may be obtained by running tests in a statistics software such as JMP on data entered in the software and performing an ANOVA analysis and/or a Spearman's rank correlation. A user may then analyze the ANOVA results to ensure a p-value of less than 0.01 or 0.05, which may vary accordingly to one skilled in the art of statistics, dependent on sample size. A user may then analyze a Spearman's rank correlation to obtain pre-specified r-values, related to the sample or samples.

Referring now to FIGS. 1 and 4, following the process of testing a concentration level or levels of an off-flavor or off-flavors in either one or multiple samples 20, a user may then determine if the tested concentrations are within limits 30. Limits may be defined as a concentration level or concentration levels of at least one compound that specify a limit or limits in which a prospective RAS may operate at, to not have its aquaculture seriously affected by the prospective at least one compound. These limits, which may also be referred to as levels, may be thought of in three types. The first may be thought of as a safe limit, the next level may be thought of as a warning limit, and the third level may be thought of as a critical level. A safe limit may be defined as a concentration of at least one off-flavor that will not noticeably affect the flavor profile of the aquaculture. A warning limit may also be defined as a concentration of at least one off-flavor that will not noticeably affect the flavor profile of the aquaculture, but may be nearing a quantifiable value that may noticeably affect the flavor profile of the aquaculture. Lastly, a critical level may be defined as a concentration of at least one off-flavor that will noticeably affect the flavor profile of the aquaculture. These limits may also be described in terms of location. By way of non-limiting example, if a portion of the RAS was tested and the test yielded a critical level of concentration, this portion may be thought of as a critical level location. The description of the limits in terms of location can be applied to all limits, safe, warning, or critical as described in the non-limiting example above.

At least one advantageous result of determining if the tested concentrations are within concentration limits 30, is to ensure the aquaculture will retain their flavor profile and ensure a prospective RAS will continue functioning as designed.

As a single, or multiple samples can comprise multiple different substances, such as, but not limited to water, aquaculture tissue, or sludge, concentration limits may be defined differently for each distinct substance. By way of non-limiting example, if water is tested, concentration limits, further defined as safe limits pertaining to the off-flavors of Geosmin and MIB may be, less than 5 nanograms of Geosmin per liter of water and/or less than 15 nanograms of MIB per liter of water. Also, by way of non-limiting example, if fish tissue is tested, concentration limits, further defined as safe limits pertaining to the off-flavors of Geosmin and MIB may be 200 nanograms of Geosmin per kilogram of fish tissue and 500 nanograms of MIB per kilogram of fish tissue.

Once a user has determined if a concentration or concentrations of a sample or samples fall within concentration limits by virtue of procedure 30, and subsequently, will have deemed a sample or samples to be at safe limits, warning limits, and/or critical limits, a user may then decide between two preferred embodiments to continue the inventive method. Both embodiments start by matching a concentration's sample to the location sampled in the RAS 32 (also 32′ per an alternative embodiment). Essentially, a user will match by virtue of identifying the location or locations where the tested sample or samples was/were taken from about the RAS. In the event that a user in procedure 30 realized that the concentration of a sample or samples was within safe limits, then a user may follow the preferred embodiment as shown in in FIG. 4 pertaining to procedure 32 and subsequently name the location the sample was sampled at as a safe limit location. In the event that a user in procedure 30 saw that the concentration of a sample or samples was not within safe limits or may have deemed the concentration was within warning limits or critical limits then a user may follow the preferred embodiment as shown in FIG. 4 pertaining to procedure 32′ and subsequently name the location the sample was sampled from as a warning limit location or critical limit location respectively. As multiple samples can be taken in earlier procedures of the inventive method, a user may carry out both embodiments simultaneously if at least one of multiple samples differs from at least one other sample in being within the described limits.

Regardless of which embodiment a user takes, or if a user decides to take both, following procedures 32 and 32′ the user will engage in designating at least one content of the aquaculture system to an appropriate area. This designation may also be known as regulating the RAS. This action of regulating may also be defined as controlling or maintaining the process of the RAS such that it operates per user inputs/desires.

Following procedure 32, a user may regulate the RAS by designating the aquaculture associated with within safe limit location(s) in the RAS 50 or designating aquaculture to remain in the RAS as the aquaculture would normally be placed without intervention. This procedure regulates the RAS by virtue of retaining the aquaculture in their prospective locations that is the location the aquaculture were in before testing, or the location the aquaculture were following testing and procedure 32. This procedure 50 may then be followed by allowing the RAS and associated locations tested to continue operating normally 52. A user allowing the RAS to continue functioning as normal may be defined as, a user carrying out the normal associated processes, methods, mitigating off-flavor buildup (as will be discussed below) or otherwise standard operating procedures of the prospective RAS, including portions of this methodology. Upon allowing the RAS to continue functioning as normal 52, the present invention may be restarted or come to an end.

Following procedure 32′, a user may regulate the RAS so as to mitigate off-flavor build up in the contents of the aquaculture system and/or preserve optimal flavor of the aquaculture farmed by the system. In one embodiment, a user may regulate the location of the aquaculture associated with a non-safe limit location 40. In another embodiment, a user may regulate the water aquaculture are in contact with at a non-safe limit location 40.

In the event that procedure 32′ was taken and aquaculture were associated with critical limit locations, the aquaculture may be subject to non-desired flavor profiles. Then, a user may decide to isolate the aquaculture to depuration 41′ by means of transporting the aquaculture from a prospective location with the RAS to an isolation tank. Transporting the aquaculture may take place, but not be limited to, netting aquaculture then releasing aquaculture in an isolation tank. An isolation tank may be filled with taint free water, and also conform to the type of water required by prospective aquaculture. This conforming of water may mean the isolation tank is filled with salt, fresh, or brackish water. The aquaculture will then remain in the isolation tank for at least a period of one day, up to three weeks. At least one advantageous result of this process is to purge aquaculture of any off-flavoring the aquaculture may have absorbed into their anatomical tissue. Subsequently, this purging may allow for the aquaculture's prospective flavor profile to return to a natural state, or state unaffected by off-flavorings.

Upon aquaculture remaining in the isolation tank for a specified period of time, a user, or group of taste-testers may then sample the prospective aquaculture for off-flavorings 42′. Such sampling may take place via compound analysis, or a group of taste testers, may consume the aquaculture such to ensure a flavor profile 42″. Upon determination and results of the sampling, the aquaculture may be left in depuration, moved to a slaughter for harvesting, or placed back in a prospective RAS. Although there is an intermediate procedure following procedure 42′ (as will be discussed below), after procedure 42″, a user may then end or restart the inventive method.

In the event that procedure 32′ was taken and aquaculture were associated with warning limit locations or critical limit locations, the aquaculture may not be at risk to be subject to non-desired flavor profiles, or be subject to non-desired flavor profiles, and a user should be aware that the warning limit could arise to a critical limit at any time. As such, a user could place aquaculture associated with a warning limit or critical limit location in a safe limit location of the RAS 41. By way of non-limiting example, this may be carried out by virtue of moving the aquaculture from one location (as will generally be associated with a warning limit location) to an alternate location of the RAS (as will generally be associated with a safe limit location). Dependent on the type of limit location the aquaculture was associated with, the aquaculture may be subject to holding in the safe limit location for a predetermined period of time.

In an alternative embodiment, and in the event that procedure 32′ was taken and aquaculture were associated with critical or warning limit locations, once again, the aquaculture may be subject to non-desired or near non-desired flavor profiles. Then, a user may decide to alter the water associated with the aquaculture at such a tested location 40. This procedure may be carried out, but not be limited to pumping in fresh, off-flavor-free water from an external source into the prospective tested location, and/or diverting water from a safe limit location of the aquaculture system to the prospective tested location. In the non-limiting example above, the outcome would be to dilute the out of concentration limit location with water that does not contain off-flavors. This would be so as to ensure subsequent steps could be carried out.

By way of non-limiting example, a combination of the two scenarios may also be performed. Aquaculture may be moved from a critical limit location or warning limit location to another critical limit location or warning limit location, that has previously had fresh, off-flavor-free water pumped in to the prospective location.

As there are generally many portions of an RAS that contain many distinct groups of aquaculture, a user may also decide a singular or groups of aquaculture located in one portion of the RAS may be moved elsewhere, wherein another singular or group of aquaculture remain in the RAS as normal, vice versa, or any combination thereof.

Regardless as to if procedure 41 or 41′ is taken, a user will then target associated off-flavoring producing sources 42. The procedure of targeting associated off-flavoring producing sources may concurrently be carried out upon a user moving the aquaculture from a first location to an alternate location of the RAS 41, such as a safe limit location and/or isolating the aquaculture to depuration 41′ by virtue of placing the aquaculture in a depuration tank, or after those procedures. Targeting associated off-flavoring producing sources may be defined as identifying compound producing sauces, which are generally interworking of an RAS. The act of targeting may also be facilitated by virtue of matching. Wherein, upon a sample being taken and subsequently tested, once the test has yielded concertation limit results, the sample and the results can then be matched back to the location it was taken at, and a user may then gain more insight as to what may be producing, if any, off-flavors. A user may gain more insight as to what may be producing off-flavors dependent on the location the sample had been matched to. By virtue of non-limiting example, if a user determined a sample to be associated with a critical limit location, and that location was an on-growing tank, a user may utilize a pre-fabricated list that indicates interworking(s) associated with that on-growing tank.

Once a user has targeted and identified what off-flavoring producing sources exist within the RAS, a user may then remedy the targeted sources from producing off-flavorings.

By way of non-limiting example, a user may remedy the targeted sources from producing off-flavorings by, draining water in tanks within the RAS and sterilizing such tanks, flushing and/or backwashing an interworking of the RAS (or limiting such an action), such as a pump, denitrification unit, biofilter, trickling filters, or drum filters and/or improving nitrification rates of the RAS to reduce ammonium levels. These acts of remedying may be correlated or directly associated with certain times and/or frequencies in which the RAS is operating. To illustrate this fact by way of non-limiting example, a user of the RAS may remedy the RAS at a specified time before or after a process of on-growing, harvesting or otherwise farming the aquaculture in the RAS.

In one embodiment, the user may have aquaculture transferred from a first tank in the RAS to a second tank in the RAS. Upon this transferring, a user may then drain the water in the first tank by virtue of syphoning off the first tank's water supply, aerating the tank with atmospheric air, and sterilize the tank by virtue of removing any residue and/or water residue, then slowly re-introducing water into the tank without the process of draining and sterilizing the first tank affecting the second.

In another embodiment, a user may limit the flushing of an interworking of the RAS and/or adjust the frequency of backwashing an interworking of the RAS. This action may be completed as it has been discovered that off-flavorings congregate or rapidly multiply on or near one or more of the above specified interworking. In this embodiment, such an action may be carried out before slaughter of aquaculture.

In yet another preferred embodiment, the user may alter the rate at which the RAS oxidizes ammonia to nitrite (nitrification rate). This action may be completed as it has been discovered that increases in off-flavorings correlate directly to ammonium levels in a prospective RAS. Thus, a user may increase or decrease nitrification rates of the RAS at certain times dependent on the farming cycle of aquaculture so as to ensure control over off-flavorings or off-flavoring production in the system.

At least one advantageous result of this is in of the targeted sources from producing off-flavorings is that upon remedying, aquaculture then placed in the RAS will be less disposed to off-flavorings, and subsequently having their prospective flavor profile compromised.

Following a user remedying the targeted sources from producing off-flavorings, a user may then ensure that the targeted sources will not produce off-flavorings again 44. This procedure will be completed as preventative maintenance. Wherein the preventative maintenance can be described as a method and/or procedure that a user of the RAS carries out on a one time, or regular basis.

By way of non-limiting example, one method by which a user may ensure that the targeted sources will not produce off-flavorings again, may follow targeting the source of biofilters and denitrification units as off-flavoring producing sources. To ensure these sources will not produce off-flavorings again, a user may avoid or limit cleaning of the biolfilters and denitrification units prior to a harvesting of the aquaculture held within a prospective RAS to prevent potential uptake of off-flavorings.

By way of another non-limiting example, one method by which a user may also ensure that the targeted sources will not produce off-flavorings again, may follow targeting the sources of an off-flavoring producing source to be an interworking of the RAS. A user may then divert flushing flows away from the RAS in regular intervals and replace water with clean makeup water and adjust the frequency of cleaning and backwashing of biofilters and denitrification units so as to prevent off-flavoring build up.

By way of yet another non-limiting example, another method by which a user may also ensure that the targeted the target sources will not produce off-flavorings again, may be to improve the nitrification rate to reduce ammonium levels which appear to correlate with a high off-flavoring producing quality.

Referring now to FIG. 7, which, as should be apparent, have elements taken from and applied to embodiments such as those described in FIGS. 5 and/or 6 and recognizing the inventive methodology of FIG. 7 may also embody a process absent any above disclosure referring to FIGS. 1-6. In one embodiment, a user may be in the presence of an RAS and simply use olfactory means to determine that the RAS contains an off-flavor, which may give way to a user determining to begin the process as described in FIG. 7. Also, a user may observe a film of biofilm resting on the surface of any water in an RAS to determine that off-flavors may be present to being to the process as described in FIG. 7. Regardless, a user may decide to utilize the process described in FIG. 7 as a standard operating procedure for an RAS or simply as a means to perform inspection of a prospective RAS. A user may decide to begin the inventive methodology by collecting a sample or samples of matter in the RAS 60. A user may choose a single location or multiple locations of a recirculating aquaculture system to carry out procedure 60. Once procedure 60 has been completed, the user may choose to indicate and/or record which location or locations of the respective RAS has been sampled from. Indication and/or recording of which location or locations of the RAS has been selected for sampling may be recorded by pen and paper, computer table entry, or by automated means.

Following completion of procedure 60, the user may then cultivate and/or isolate microbiota from the sample or samples of matter 62. Microbiota may be defined as any living organisms of a particular site. Procedure 62 may be carried out in a controlled environment or multiple controlled environments where factors such as air temperature, humidity, pressure, heat indexes, or other environmental factors are predetermine or set. Cultivation or isolation of the microbiota from the sample or samples of matter may be further progressed by the methods of culturing such as, but not be limited to, culture media, pure culturing, streak culturing, lawn culturing, stroke culturing, stab culturing, and/or pour plate method culturing. In one embodiment, cultivation and/or isolation may be carried out by placing the sample or samples of matter on one or multiple carrier discs. The one or multiple carrier discs may then be placed in a temperature controlled environment, of negative (−) 80 degrees Celsius for subsequent analysis. The samples may then be thawed or unthawed and filtered using filter. Such filters may be 0.2 micro meter membrane filters, which may be produced by Whatman from Dassel, Germany. Filtration may be performed so as to obtain cultured or isolated microbiota. In the event that the 0.2 micro meter membrane filters were utilized, the filters would be utilized fresh and frozen. Regardless of isolation and/or cultivation methodology used, the end product of procedure 62 will be microbiota in absence of any other non-living substance from the sample or samples of matter. As one sample, or multiple samples may be taken, procedure 62 may yield one or multiple cultures and/or isolations of microbiota.

Following procedure 62, a user may then extract the DNA(s) and/or RNA(s) of the microbiota from the sample or samples of matter 64. As should be inherent, the microbiota may comprise a plethora of living organisms, each with unique DNA(s) and/or RNA(s). Procedure 64 may be utilized in order to obtain all genetic material from each individual organism of the microbiota. In one embodiment, 64 may be carried out by virtue of collecting one or multiple microbial cultures, and/or placing the culture or cultures in a tube or tubes, such as, but not limited to a falcon tube(s) and/or sterilized tube(s) comprised of plastic and/or glass. The tube or tubes may then be subject to centrifugation for a pre-determined time for a predetermined revolution per time rate per mass of substance held within the tube or tubes. Following centrifugation, it is common for a single supernatant or multiple supernatants to form or become more prevalent. The supernatant or supernatants may then be removed, and the tube or tubes may be subject to one or more multiple rounds of centrifugation for a pre-determined time for a predetermined revolution per time rate per mass of substance held within the tube or tubes. If the tube or tubes are subject to one or more multiple rounds of centrifugation, after each centrifugation, a user may remove a supernatant or supernatants formed.

The final substance in the tube or tubes may then be utilized for extraction of genetic material. In one embodiment, a FastDNA-96™ Fecal DNA Kit with Matrix E produced by MP Biomedicals, USA may be utilized for lysis so as to produce lysates from the final substance in the tube or tubes. In another embodiment, lysis is applied to the final substance produced in the tube or tubes so as to produce lysates. The lysates of either embodiment may then have a purification agent introduced into the tube, tubes, or environment in which the lysates reside, had they been transferred from the tube or tubes. In another embodiment, the lysates of either embodiment may then have a purification process applied to the tube, tubes, or environment in which the lysates reside, had they been transferred from the tube or tubes. The end result of the procedure 64 may then result in a user obtaining genetic material, which may be DNA(s) or RNA(s) of the microbiota from the sample or samples of matter.

Following completion of procedure 64, a user may then sequence the DNA(s) or RNA(s) of the microbiota 66. The DNA(s) or RNA(s) may be sequenced utilizing a DNA or RNA sequencing methodology paired with the appropriate machinery and/or software, of which may be, but not be limited to CRISPR, deep sequencing, qPCR, ILLUMINA, Quant-iT, and/or PacBio. In one embodiment, all DNA(s) extracted from the microbiota may be subject to deep sequencing. The deep sequencing may involve paired end sequencing of regions of DNA(s) that are conserved and or hypervariable regions. In another embodiment, the deep sequencing may involve paired end sequencing of DNA(s) suitable for taxonomic classifications. Further, polymerase chain reaction (PCR) may be performed at least one time per DNA(s) sample by targeting regions by virtue of including Illumina flowcell adapter sequences to DNA(s) samples. DNA(s) samples may be purified with Ampure before and/or after PCR so as to quantify the samples using the Quant-iT Picogreen ds DNA(s) with picogreen. The DNA(s) samples may then be sequenced on a MiSeq wherein the MiSeq contains a control library from PhiX Control to account for reading. Regardless of the DNA(s) sequencing methodology used, even when paired with the appropriate machinery and/or software, the end result of procedure 66 is sequenced DNA(s) of the microbiota.

Following procedure 66, a user may then quantify and analyze data produced from sequenced DNA(s) or RNA(s) 68. It may be commonly known to one skilled in the art, following sequencing DNA(s), sequenced DNA(s)'s function and characteristics cannot be understood without at least the process of annotation to the sequenced DNA(s). In one embodiment, the DNA(s) sequenced from procedure 66 may be processed utilizing an open sourced bioinformatics library, such as, but not be limited to, Quantitative Insight Into Microbial Ecology (QIIME) so as to annotate the DNA(s). DNA(s) sequence(s) may be demultiplexed and checked against a quality score within QIIME so as to ensure a correctness of processing and subsequently annotation. Intra QIIME, OTU identities may be assigned to a taxa, where the taxonomic level assigned can vary from phylum, class, order, gamily, genus and species level. Within this processing, OTU identities can be assigned to the same taxa. Taxa may then be used to create an OTU table that may further be analyzed via utilization of within-DNA(s) sample diversity, between-DNA(s) sample diversity, and filtering to subsequently allow for taxonomic analysis and identification. In the embodiment wherein the OTU table is further analyzed, the OTU table may be analyzed to ensure creation of the table is accurate. Analyzation by virtue of statistical analysis may then be performed. Statistical analysis may be carried out by virtue of multiple linear regression wherein two way ANOVA tables may be produced so as to test interactions of within-DNA(s) sample diversity and between-DNA(s) sample diversity. Further, partial least squares regression may be used to ensure stability of predictions and/or temporal chances of OTU identification shown in the OTU table.

Following taxonomic analysis, the DNA(s) may then be realized for function and location of a specific coding region in a genome. This realization gives way so the taxonomic analysis may be utilized for annotation. This annotation may then allow for identification of living organisms to be realized. This realization may be used in identifying living organisms on the phylum and subsequently, the genus level, of which the following may be, but not be limited to identification of Streptomyces, Kitasotospora, Frankia, Saccharopolyspora, Nocardia, Actinosynnema, Micromonospora, Actinomycetales spp, Oscillatoria, Lyngbya, Phormidium, Schizothrix, Anabaena, Microcoleus, Aphanizomenon, Planktothrix, Symploca, Calothrix, Cylindrospecrmum, Nstoc, Pseudanabaena, Synechococcus, Myxococcus, Myxococcales, Stigmatella, Nannocystis, and/or Sorangium, all of which are commonly known or theorized to produce geosmin or MIB. The identification of the living organism may be of one or multiple living organisms.

As commonly known, genus comprises a further sub-taxonomy of species. Following the identification of at least one living organism to be realized by annotation, at least one genome alignment may then be constructed on the annotated DNA(s). The at least one genome alignment may result in at least one genome comprising the genus of one of the above mentioned living organisms, with a near infinitely possible species identifier. The at least one genome alignment may also result in new discoveries of phylum, genus, or species that can be identified or hypothesized to produce Geosmin or MIB. Regardless as to the specific benefits that at least one genome alignment would yield, the at least one genome alignment could provide a user with the understanding as to the phylum, genus, species and quantified genetic codes of the microbiota. Further, many genome alignments may also be yielded, giving way to providing a user with an understanding as to all the multiple phylum, genus, species and quantified genetic codes of all living organisms of the microbiota.

Upon discovery and realization of the phylum, genus, species and quantified genetic codes (singularly or in plurality of which may be known as genetic material) of the microbiota, a user may then be able to engineer at least one bacteriophage therapy from the data produced 70. The phylum, genus, species and quantified genetic codes of the microbiota may yield a range of living organisms that are commonly known, living organisms commonly known to produce the off-flavors of Geosmin and/or MIB, living organisms not commonly known to produce the off-flavors of Geosmin and/or MIB, and/or living organisms not commonly known. The common knowledge of such living organisms may be discovered and/or realized in a biological database, such as a bioinformatics library.

A user may then wish to begin carrying out a biofoundry methodology or approach given the range of living organisms yielded so as to begin engineering at least one bacteriophage therapy from the data produced 70. A biofoundry methodology/approach may be defined as at least a multi-tiered method able to bend or shape biology in order to serve a certain need or reach a goal. As should be inherent, one of the advantages of implementing such a biofoundry approach/methodology is to reach a goal of engineering at least one bacteriophage therapy for an aquaculture system.

In one embodiment, a portion of the biofoundry approach may comprise a user accessing a biological database, inputting the phylum, genus, species, and/or quantified genetic codes of the microbiota (wherein multiple living organisms may comprise the microbiota) so as to attempt to identify at least one bacteriophage infectious of the microbiota's genetic material, and subsequently, the living organisms thereof. A biological database a user may use may be a bioinformatics library, or database comprising genome sequences of bacteriophages. If a user has been able to identify at least one bacteriophage infectious of the microbiota's genetic material, a user may identify the genome sequence(s) of the at least one identified bacteriophage. The genome sequence identified may also comprise at least one quantified nucleotide sequence, wherein the at least one quantified nucleotide sequence is combative of the microbiota's genetic material.

In another embodiment, a portion of the biofoundry approach may also comprise a user accessing a biological database, inputting the phylum, genus, species, and/or quantified genetic codes of the microbiota so as to attempt to identify at least one bacteriophage infectious of the microbiota's genetic material. A user may not be able to identify such a bacteriophage infectious of the microbiota's genetic material, by way of non-limiting example, if the biological database does not contain information on the microbiota's genetic material, or at least a portion of the microbiota's genetic material is not commonly known. In such an embodiment, a user may wish to carry out further portions of a biofoundry method so as to identify at least one genome sequence of at least one bacteriophage combative of the microbiota's genetic material, wherein such portions may be defined as, but not be limited to, simulations of DNA(s) designs/synthesis, simulations of genome and plasmid engineering, simulations of enrichment of biological systems, and/or simulations of bioinformatics analysis modeling, wherein simulations may be automated or user conducted. If a user is able to identify at least one bacteriophage genome infectious of the quantified microbiota's genetic material, a further realization of the combative bacteriophage's characteristics may be able to be obtained through subsequent testing, wherein such characteristics may be, but not be limited to a quantified nucleotide sequence combative, or theorized to be combative of the quantified microbiota's genetic material.

Upon a user obtaining at least one quantified nucleotide sequence that is combative, or theorized to be combative of the quantified microbiota's genetic material, wherein the at least one quantified nucleotide sequence belongs to at least one bacteriophage infectious of the microbiota's genetic material, the user may continue to engineer at least one bacteriophage therapy from the data produced 70. To further this procedure (70), a user may further progress a step or portion of the biofoundry methodology. To further progress a step and/or portion of the biofoundry methodology, a user may take the at least one quantified nucleotide sequence that is combative, or believed to be combative and/or bacteriophage genome infectious of the quantified microbiota's genetic material, and apply a bacteriophage engineering methodology, which may be, but not be limited to, a Workbench for Virus Design (ETH Zurich), adjustment of a host for a bacteriophage (Russel & Bikard), homologous recombination, recombineering of electroporated DNA(s), CRISPR-Cas-Based phage engineering, and/or rebooting phages using assembled phage genomic DNA(s) to the nucleotide sequence and/or genome so as to produce at least one bacteriophage. Any singular or combination of these methodologies may utilize and/or subsequently produce, but not be limited to at least one wild, modified, and/or synthetic seed stock bacteriophage. As many quantified nucleotide sequences that are combative or believed to be combative of the quantified microbiota's genetic material may be discovered, many bacteriophages may be produced. Also, as many bacteriophage genomes infectious of the quantified microbiota's genetic material may be discovered, many bacteriophages may be produced. At production, at least one bacteriophage may be produced that specifically infects at least one of the living organisms identified in the microbiota that are known to, or hypothesized to produce off-flavorings. In an alternative embodiment, many bacteriophages may be produced that specifically infect all of the living organisms identified in the microbiota that are known to, or hypothesized to produce off-flavorings.

Upon production of the at least one bacteriophage, a further step and/or portion of the biofoundry method may be applied to the procedure 70. This step may include, but not be limited to selecting at least one bacteriophage created and at least hypothesized to infect living organisms of the microbiota known or hypothesized to produce off-flavorings. This selecting may be one bacteriophage or multiple. A user may then place the selection in a bacteriophage cocktail. A bacteriophage cocktail may be defined as, but not be limited to, at least one user selected bacteriophage residing in a medium, wherein the medium may be, but not be limited to, water, saline, water mixed with dextrose, gelatin, and/or hypromellose.

Upon the creation of a bacteriophage cocktail, a user may then strategize or optimize administration of the bacteriophage cocktail to a prospective RAS wherein the sample containing the microbiota was obtained from. A user may strategize and/or optimize administration of the bacteriophage cocktail via computerized simulations, selecting a location of the RAS used in sampling, and/or selecting a location of a prospective RAS either known or hypothesized to contain off-flavors. Further, a user may strategize and/or optimize administration of the bacteriophage cocktail via selecting pre-determined times at which to introduce curated bacteriophage cocktails to the RAS. By way of non-limiting example, a user may optimize administration of the bacteriophage cocktail via determining to apply a first round of a bacteriophage cocktail, one of at least one bacteriophage at a pre-determined PFU, at a predetermined location, at a predetermined hour of a day, then a user may also determine applying a second round of another bacteriophage cocktail, one of multiple bacteriophages at a different PFU from the first round, at a different location with the RAS from the first round, and a different hour of a day from the first round. In strategizing and/or optimizing, a user may specify a PFU of any bacteriophage cocktail so as to ensure the introduction of the cocktail to a specified RAS will not become toxic to the contents of the aquaculture system. By way of non-limiting example, a user may specify a PFU with a factory of safety in-mind to ensure bacteriophages do not proliferate to a count or amount of bacteriophages that may become toxic or overloading to the contents of the RAS. Further, this specification of PFU may be strategized or optimized so as to ensure aquaculture bred within the RAS do not absorb or intake the at least one bacteriophage introduced by the bacteriophage cocktail to the point where the aquaculture have their prospective flavor profiles become less than optimal. In an alternative embodiment, the PFU strategized and/or optimized will be a low enough value such that the aquaculture will retain optimal flavor profiles from the introduction of a proposed bacteriophage cocktail. Also, the strategy and/or optimization of the administration of the bacteriophage cocktail can account for continuing to allow the RAS to function as normal, wherein normal functioning of an RAS has been previously defined.

A user may then apply the engineered bacteriophage therapy to the RAS 80. In one embodiment, this procedure (80) may be carried out by a user administering at least one bacteriophage cocktail (as produced in procedure 70) to at least one specified location of an RAS (as determined in procedure 70) at at least one specified time (as determined in procedure 70). Upon applying the engineered bacteriophage therapy to the RAS 80, a user may expect that the therapy (and subsequently the at least one bacteriophage in the therapy) infects microbiota of the RAS at least hypothesized to produce at least one off-flavor. Infection of the microbiota resulting from the bacteriophage therapy may be followed by the microbiota experiencing a lethal overdose of a bacteriophage, wherein the lethal overdose ultimately destroys the microbiota, which prohibits further growth of such microbiota. Following a lethal overdose of a microbiota, the bacteriophage may proliferate from the microbiota and continue to infect subsequent microbiota.

Following application of the bacteriophage therapy to an RAS, the process may be deemed complete, or restarted.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method of preserving optimal flavoring in aquaculture bred within an aquaculture system comprising:
taking at least one sample of water from at least one portion of the aquaculture system that contains aquaculture;
extracting genetic material from microbiota of the at least one sample;
quantifying the microbiota's extracted genetic material;
creating at least one bacteriophage in relation to the quantified microbiota's genetic material; and
introducing the at least one bacteriophage into the aquaculture system.

2. The method of claim 1, wherein the aquaculture system is a recirculating aquaculture system.

3. The method of claim 1, wherein said step of taking at least one sample of water from at least one portion of the aquaculture system, comprises taking a sample of water from an interworking of the aquaculture system.

4. The method of claim 1 wherein said step of extracting genetic material from microbiota of the at least one sample comprises cultivation of microbiota from the at least one sample.

5. The method of claim 1, wherein said step of extracting genetic material from microbiota of the at least one sample further comprises extraction of cultivated microbiota's DNA(s).

6. The method of claim 5, wherein extraction of cultivated microbiota's DNA(s) comprises centrifugation of a cultivation of microbiota mixed with a diluent.

7. The method of claim 5, wherein extraction of cultivated microbiota's DNA(s) further comprises application of lysis to a centrifugated cultivation of microbiota mixed with a diluent so as to produce at least one lysate.

8. The method of claim 5, wherein extraction of cultivated microbiota's DNA(s) further comprises purification of at least one lysate so as to obtain, then be able to extract cultivated microbiota's DNA(s).

9. The method of claim 1, wherein said step of quantifying the microbiota's extracted genetic material comprises sequencing the microbiota's extracted DNA(s).

10. The method of claim 9, further comprising annotating the sequenced DNA(s).

11. The method of claim 10, comprising performing statistical analysis on the DNA(s) so as to ensure correct results of annotation.

12. The method of claim 11, further comprising constructing at least one genome alignment from the annotated, sequenced DNA(s).

13. The method of claim 1, wherein said step of creating at least one bacteriophage in relation to the quantified microbiota's genetic material comprises identification of at least one phage genome sequence commonly known to infect the quantified microbiota's genetic material.

14. The method of claim 13, wherein identification of at least one phage genome sequence commonly known to infect the quantified microbiota's genetic material is identified via utilization of at least one biological database.

15. The method of claim 13, wherein the identified at least one phage genome commonly known to infect the quantified microbiota's genetic material comprises at least one quantified nucleotide sequence.

16. The method of claim 15, further comprising producing at least one quantified nucleotide sequences of at least one phage genome commonly known to combat the quantified microbiota's genetic material.

17. The method of claim 16, further comprising applying a biofoundry methodology to the at least one quantified nucleotide sequence of at least one phage genome so as to create at least one bacteriophage.

18. The method of claim 17, wherein at least one portion of the biofoundry methodology applied comprises the bacteriophage engineering methodology of at least one of, homologous recombination, recombineering of electroporated DNA(s), CRISPR-Cas-Based phage engineering, and rebooting phages using assembled phage genomic DNA(s).

19. The method of claim 1, wherein the at least one bacteriophage introduced into the aquaculture system infects microbiota known to produce at least one off-flavor compounds so as to destroy the microbiota thereby prohibiting further growth of the microbiota infected.

20. The method of claim 19, wherein the at least one off-flavor compound comprises at least one of, Geosmin and 2-Methylisoborneol.

21. The method of claim 1 wherein the introduction of at least one bacteriophage allows the aquaculture system to continue functioning as normal.

22. The method of claim 1 wherein the at least one bacteriophage introduced into the aquaculture system is non-toxic to the contents of the aquaculture system.

23. The method of claim 1 wherein the aquaculture bred within the aquaculture system retain optimal flavoring from the introduction of the at least one bacteriophage in the aquaculture system.
